(19) United States
(12) Patent Application Publication
(10) Pub. No.: US 2011/0190668 A1
(43) Pub. Date: Aug. 4, 2011

(54) ULTRASOUND NEUROMODULATION OF THE SPHENOPALATINE GANGLION

(76) Inventor: David J. Mishelevich, Playa del Rey, CA (US)

(21) Appl. No.: 13/020,016

(22) Filed: Feb. 3, 2011

Related U.S. Application Data
(60) Provisional application No. 61/300,828, filed on Feb. 3, 2010.

(51) Int. Cl. A61N 7/00

(52) U.S. Cl. 601/2

(57) ABSTRACT

Disclosed are methods and systems for non-invasive neuromodulation of the Sphenopalatine Ganglion and associated neural structures vidian nerve and/or sphenopalatine nerve using an ultrasound transducer to treat migraine and cluster headaches as well as other indications such as neurologic and psychiatric conditions. Treatment may be unilateral or bilateral.

### CROSS REFERENCE TO RELATED APPLICATIONS

This patent application claims priority to provisional patent applications Application No. 61/300,828, filed Feb. 3, 2010, entitled “NEUROMODULATION OF SPHENOPALATINE GANGION USING ULTRASOUND.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation of the Sphenopalatine Ganglion and related neural structures to stimulate intracranial neural circuits.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Neural structures are usually assembled in circuits. For example, nuclei and tracts connecting them make up a circuit. The potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009)

Note that while Transcranial Magnetic Stimulation (TMS) is an effective means of non-invasive neuromodulation when used intracranial, its delivered footprint is too large for neural structures like the Sphenopalatine Ganglion. Ultrasound can be focused to 0.5 to 2 mm while TMS can be focused to 1 cm at best. Also, if TMS were used to stimulate the Sphenopalatine Ganglion there would be intolerable side effects such local muscle stimulation, and, in some cases stimulation of other nerves.

Autonomic stimulation to positively impact intracranial structures such as the Vagal Nerve Stimulation (VNS) is used successfully in clinical practice (e.g., George, M., Sackheim, A J, Rush, et al., “Vagus Nerve Stimulation: A New Tool for Brain Research and Therapy,” Biological Psychiatry, 47, 287-295, 2000).

Sphenopalatine Ganglion and other autonomic nervous system stimulation has been associated with treatment of headaches and associated symptoms such as nausea and vomiting. A variety of non-invasive treatments have been used for headache treatment such as medication, diet, avoidance of triggers, acupuncture, anesthetic agents, biofeedback, and physical therapy. Invasive treatments have been used as well such as ganglion resection, ganglion block, radiosurgery, and cryotherapy. In addition, electrical stimulation has been applied by implanted electrodes or implanted stimulator.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

In addition, stimulation of the Sphenopalatine Ganglion has been described for modification the properties of the Blood Brain Barrier (BBB) and cerebral blood flow (Shalev, A. and Y. Gross, “Method and apparatus for stimulating the sphenopalatine ganglion to modify properties of the BBB and cerbral blood flow,” U.S. Pat. No. 7,190,998, Issued Mar. 13, 2007).

The sphenopalatine ganglion is a parasympathetic ganglion the largest of the parasympathetic ganglia associated with the branches of the trigeminal nerve. Stimulation of the Sphenopalatine Ganglion (SPG) for a number of maladies has been addressed previously. Examples are Pless (B. D. Pless, “Method and Device for the Treatment of Headache,” U.S. Patent Application Pub. No. 2009/0276005), Yun and Lee (Yun, A. J., and P. Y-b Lee, “Treatment of conditions through modulation of the autonomic nervous system,” U.S. Pat. No. 7,363,076), and Ansarinia (Ansarinia, M. M., “Stimulation Method for the Sphenopalatine Ganglia, Sphenopalatine Nerve, or Vidian Nerve for Treatment of Medical Conditions,” U.S. Pat. No. 6,526,318, Feb. 25, 2003). It would desirable to have neuromodulation of the SPG and related structures without using invasive means such as implanted electrodes.

### SUMMARY OF THE INVENTION

It is the purpose of this invention to provide methods and systems and methods for ultrasound neuromodulation of the Sphenopalatine Ganglion and associated neural structures vidian nerve and/or sphenopalatine nerve. Such neuromodulation can effectively used for the treatment of migraine and cluster headaches in their multiple variations as well as a number of other conditions.

Stimulation of the Sphenopalatine Ganglion by this invention could also include the sphenopalatine nerve and/or the vidian nerve.

### BRIEF DESCRIPTION OF THE DRAWINGS

FIG. 1 shows ultrasound transducer against the face of the patient targeting the Sphenopalatine Ganglion and related neural structures.

FIG. 2 shows a diagram of the Sphenopalatine Ganglion and related neural structures.

FIG. 3 illustrates the anatomic relationships of the Sphenopalatine Ganglion and related neural structures with the bony structures of the face.

FIG. 4 shows a block diagram of the control circuit.

### DETAILED DESCRIPTION OF THE INVENTION

It is the purpose of this invention to provide methods and systems and methods for ultrasound neuromodulation of the Sphenopalatine Ganglion and associated neural structures vidian nerve and/or sphenopalatine nerve. The neuromodulation of the Sphenopalatine Ganglion and associated structures in turn neuromodulates connected intracranial structures to obtain therapeutic results.

The acoustic frequency (e.g., 0.44 MHz (typically in that range of 0.3 MHz to 0.8 MHz) that permits the ultrasound to effectively penetrate through bone) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). In most cases of neuromodulation of the Sphenopalatine Ganglion and associated structures the mode will be excitation. Power is generally applied at a level less than 60 mW/cm2. Ultrasound pulses may be monophasic or biphasic, the choice made based on the specific patient and condition. Such vendors as Blatek and Keramos-Etalon in the U.S. and Imasonic in France can supply suitable ultrasound transducers.

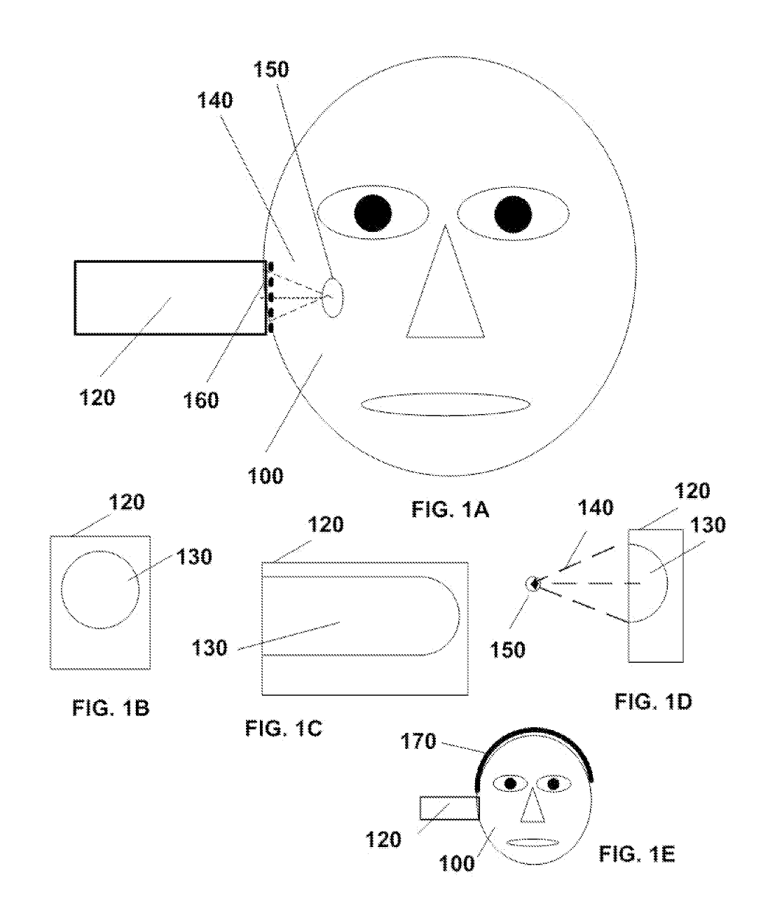

FIG. 1A shows a frontal view of the configuration for neuromodulation of the Sphenopalatine Ganglion (SPG) and related structures such as the sphenopalatine nerve and the vidian nerve. For the purpose of this discussion that the additional structures could be included. Patient head 100 contains Sphenopalatine Ganglion 150. Ultrasound transducer 120 focuses sound field 140 on Sphenopalatine Ganglion 150. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (130 in FIGS. 1B-1D). Ultrasound gel layer 160 that provides the interface for ultrasound conduction between ultrasound transducer 120 and head 100 completes the conduction pathway. In the illustrated embodiment, ultrasound transducer 120 is elongated to allow a longer focal length to be employed. The elongated shape is convenient for the patient to hold and also for use with a positioning headband as shown in FIG. 1E showing patient head 100 with ultrasound transducer 120 and headband 170. Ultrasound transducer 120 is moved in and out of a holder (not shown) to provide the appropriate distance between ultrasonic transducer 120 and Sphenopalatine Ganglion target 150. In other embodiments, alternative fixed configurations, either of ultrasonic transducer focal lengths or of different fixed positions in holders are available for selection for specific patients. As to X-Y position on the head, the treatment for a specific patient can be planned using physical landmarks on the patient (for example, positioning the ultrasound transducer at lower edge of the zygomatic arch at the point anteriorly-posteriorly where the frontal process of the zygomatic bone meets the temporal process of the zygomatic bone). Alternatively, a standard x-ray examination based on bone can be done; taking an MRI or other scan is not necessary. In addition, the patient can adjust positioning based on effect. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well.

Ultrasound transducer 120 with ultrasound-conduction-medium insert 130 is shown in front view in FIG. 1B and in a side view in FIG. 1C. FIG. 1D again shows a side view of ultrasound transducer 120 and ultrasound-conduction-medium insert 130 with ultrasound field 140 focused on the Sphenopalatine Ganglion target 150. The focus of ultrasound transducer 120 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Transducer arrays of the type 120 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Blatek and Keramos-Etalon in the U.S. are other custom-transducer suppliers. The design of the individual array elements and power applied will determine whether the ultrasound is high intensity or low intensity (or medium intensity) and because the ultrasound transducers are custom, any mechanical or electrical changes can be made, if and as required.

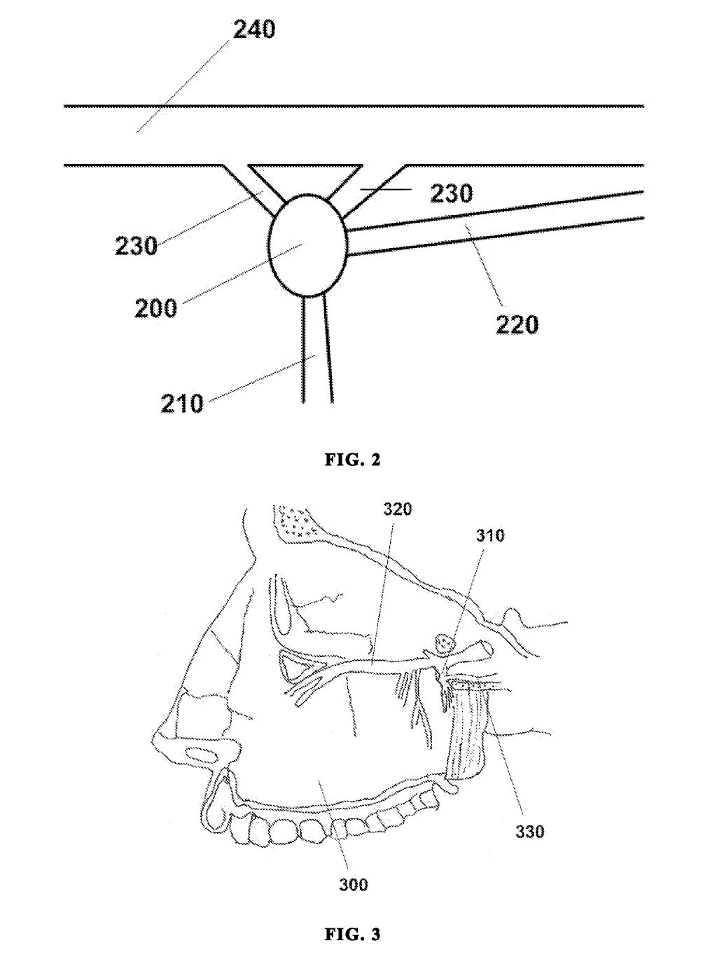

FIG. 2 shows the configuration surrounding Sphenopalatine Ganglion 200. Sphenopalatine Ganglion 200 is contained within the Sphenopalatine (or Pterygopalatine) fossa (not shown) and hangs down from maxillary nerve 240 connected to it by Sphenopalatine Nerves 230 with connections to vidian nerve 220 and palatine nerves 210. The vidian nerve 220 connects to the Sphenopalatine Ganglion 200. Vidian nerve 220 contains parasympathetic fibers (which synapse to Sphenopalatine Ganglion 200). The vidian nerve also contains sympathetic fibers and sensory fibers, transmitting sensation from part of the nasal septum. The sphenopalatine nerves 230 are sensory nerves physically connect the Sphenopalatine Ganglion 200 to the maxillary nerve 240, but pass through and do not synapse with Sphenopalatine Ganglion 200. These structures are located bilaterally. Neuromodulation of which side will be most effective is headache specific and patient specific. In an alternative embodiment, bilateral neuromodulation will be supplied. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well. FIG. 3 shows selected physical relationships with anterior skull 300 showing Sphenopalatine Ganglion 310, maxillary nerve 320, and vidian nerve 330.

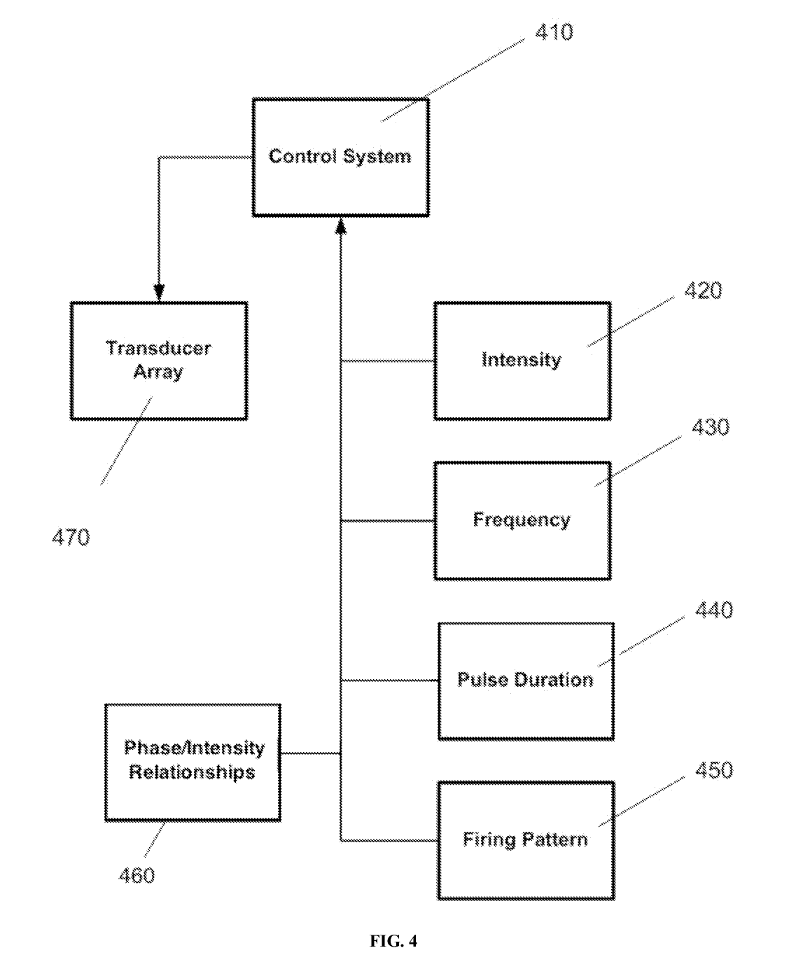

FIG. 4 illustrates the control circuit. Control System 410 receives its input from Intensity setting 420, Frequency setting 430, Pulse-Duration setting 440, Firing-Pattern setting 450, and Phase/Intensity Relationships 460. Control System 410 then provides output to drive Transducer Array 470 and thus deliver the neuromodulation. Settings may be input by the healthcare professional or, under the prescription and directions of a physician, set by the patient.

While the parasympathetic nervous system is subject to Long-Term Potentiation (LTP) such that in addition to the acute effect that there is the potential for a long-term training effect, there can be Long-Term Potentiation (LTP) and Long-Term Depression (LTD) at the intracranial targets to which the Sphenopalatine Ganglion and associated neural structures are attached.

The invention can be applied to a number of conditions including headaches in various forms, migraine headaches in various forms, cluster headaches in various forms, neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders. It can also be applied to modification of the properties of the blood-brain Barrier and cerebral blood flow.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A method of non-invasively neuromodulating the target Sphenopalatine Ganglion and associated structures using ultrasound stimulation, the method comprising:
aiming an ultrasound transducer at the target,
applying pulsed power to said ultrasound transducer via a control circuit thereby modulating the activity of the target,
whereby connected intracranial neural structures are neuromodulated.

2. The method of claim 1, wherein the plurality of control elements is selected from the group consisting of intensity, frequency, pulse duration, firing pattern, and phase/intensity relationships.

3. The method of claim 1, further comprising focusing the sound field of an ultrasound transducer at the target Sphenopalatine Ganglion and associated structures neuromodulating the activity of the target in a manner selected from the group of up-regulation, down-regulation.

4. The method of claim 1, wherein the acoustic ultrasound frequency is in the range of 0.3 MHz to 0.8 MHz.

5. The method of claim 1, where in the power applied is less than 60 mW/cm².

6. The method of claim 1, wherein the configuration of ultrasound power is selected from the group consisting of monophasic and biphasic.

7. The method of claim 1, wherein a stimulation frequency for of 300 Hz or lower is applied for inhibition of neural activity.

8. The method of claim 1, wherein the stimulation frequency for excitation is in the range of 500 Hz to 5 MHz for excitation of neural activity.

9. The method of claim 1, wherein the focus area of the pulsed ultrasound is 0.1 to 0.5 inches in diameter.

10. The method of claim 1, wherein the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

11. The method of claim 1, wherein the neuromodulation of the Sphenopalatine Ganglion and related neural structures is selected from the group consisting of unilateral and bilateral.

12. The method of claim 1, wherein the neuromodulation results in a durable effect selected from the group consisting of Long-Term Potentiation and Long-Term Depression.

13. The method of claim 1, wherein the disorder treated is selected from the group consisting of headaches in various forms, migraine headaches in various forms, cluster headaches in various forms, neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

14. The method of claim 1 wherein ultrasound mediated modification is selected from the group consisting of the properties of the Blood-Brain Barrier and cerebral blood flow.

15. The method of claim 1, wherein ultrasound therapy is combined with one or more therapies selected from the group consisting of medications, electrical stimulation, local anesthetic blocks, surgical transection, surgical resection, radiofrequency, alcohol/phenol infiltration, radiosurgery, cryotherapy, medication, avoidance of triggers, diet modification, physical therapy, chiropractic manipulation, and acupuncture.
